(19) United States
(12) Patent Application Publication
(10) Pub. No.: US 2002/0102246 A1
(43) Pub. Date: Aug. 1, 2002

(54) Antimicrobial compositions

(75) Inventors: Palle Schneider, Lynge (DK); Soren Moller, Holte (DK); Kirsten Biedermann, Horsholm (DK); Charlotte Johansen, Holte (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/850,316

(22) Filed: May 7, 2001

Related U.S. Application Data
(60) Provisional application No. 60/204,710, filed on May 16, 2000.

(30) Foreign Application Priority Data
May 8, 2000 (DK) PA 2000 00755

(51) Int. Cl. A61K 38/44; A61K 7/00

(52) U.S. Cl. 424/94.4; 424/401; 510/320

(57) ABSTRACT

The present invention relates to an enzymatic method for killing or inhibiting microbial cells or microorganisms, e.g. in laundry, on hard surfaces, in water systems, on skin, on teeth or on mucous membranes. The present invention also relates to the use of said enzymatic composition for preserving food products, cosmetics, paints, coatings, etc.

### CROSS-REFERENCE TO RELATED APPLICATIONS

This application claims, under 35 U.S.C. 119, priority or the benefit of Danish application no. PA 2000 00755 filed May 8, 2000 and U.S. application no. 60/204,710 filed May 16,2000, the contents of which are fully incorporated herein by reference.

### FIELD OF THE INVENTION

The present invention relates to an enzymatic antimicrobial method for killing or inhibiting microbial cells or microorganisms present, e.g., in laundry, on hard surfaces, in water systems, on skin, on teeth or on mucous membranes. The present invention also relates to the use of an enzymatic antimicrobial composition for preserving food products, cosmetics, paints, coatings, etc.

### BACKGROUND

Various enzymatic antimicrobial compositions are known in the art. For instance, WO 94/04127 discloses stabilized dentifrice compositions which are capable of producing antimicrobially effective concentrations of hypothiocyanite ions. The compositions contain an oxidoreductase capable of producing hydrogen peroxide and a peroxidase enzyme capable of oxidizing thiocyanate ions normally present in saliva to antimicrobial hypothiocyanite ions. Suitable peroxidases include lactoperoxidase, myeloperoxidase, salivary peroxidase and chloroperoxidase.

The object of the present invention is to provide an enhanced method for killing or inhibiting microbial cells.

### SUMMARY OF THE INVENTION

According to the present invention there is provided a method for killing or inhibiting microbial cells comprising treating said microbial cells with a phenol oxidizing enzyme system and an enhancing agent selected from the group consisting of:

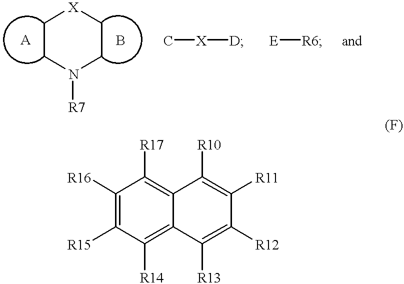

in which C, D, and E independently of each other are:

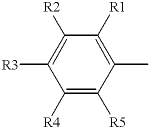

and R1, R2, R3, R4, R5, R6, R7, R10, R11, R12, R13, R14, R15, R16, R17 independently of each other are H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl; and X is a single bond or NH, NCH₃, NC₂H₅, O, S, N═N, CH═N, or CH═CH.

A and B are 6 membered aromatic rings, and may independently of each other be substituted with H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl.

One or more carbon atoms of the aromatic rings of A, B, C, D, and E may independently of each other be substituted with N or S, thus rendering said aromatic ring a heterocyclic ring.

In further aspects, the present invention relates to methods for killing or inhibiting microbial cells in laundry, in cosmetic products or on hard surfaces.

In still further aspects, the present invention relates to use of an enzymatic antimicrobial composition for cleaning of contact lenses, for cleaning of water systems, for preserving of paint, and in a cleaning-in-place system.

### DETAILED DESCRIPTION

In the context of the present invention the term “antimicrobial” is intended to mean that there is a bactericidal and/or a bacteriostatic and/or fungicidal and/or fungistatic effect and/or a virucidal effect and/or a sporicidal effect, wherein

The term “bactericidal” is to be understood as capable of killing bacterial cells.

Bactericidal activity is measured as a logarithmic reduction (log reduction) in the number of living cells or Colony Forming Units pr. ml (CFU/ml), e.g. 1 log reduction corresponds to a reduction in the number of living cells of Pseudomonas putida ATCC12633 from Y×10^{X} CFU/M (CFU: Colony Forming Units; M: ml or g) to Y×10^{X-1} CFU/M, where X can be 1, 2, 3, 4, 5, 6, 7, 8, 9, 10 or 11, and Y can be any number from 0 to 10. The number of living cells are to be determined as the number of Pseudomonas putida ATCC12633, which can grow on LB Agar (#0285, Merck, Germany) plates at 30° C.

The term “bacteriostatic” is to be understood as capable of inhibiting bacterial growth, i.e. inhibiting growing bacterial cells.

The term “fungicidal” is to be understood as capable of killing fungal cells.

The term “fungistatic” is to be understood as capable of inhibiting fungal growth, i.e. inhibiting growing fungal cells.

The term “virucidal” is to be understood as capable of inactivating virus.

The term “sporicidal” is to be understood as capable of inactivating spores.

The term “microbial cells” denotes bacterial or fungal cells, and the term “microorganism” denotes a fungus (including yeasts) or a bacterium.

In the context of the present invention the term “inhibiting growth of microbial cells” is intended to mean that the cells are in the non-growing state, i.e., that they are not able to propagate.

The “phenol oxidizing enzyme system” describes an enzyme possessing peroxidase activity together with a hydrogen peroxide source, or a laccase or laccase related enzyme together with oxygen.

The term “hard surface” as used herein relates to any surface, which is essentially non-permeable for microorganisms. Examples of hard surfaces are surfaces made from metal, e.g., stainless steel, plastics, rubber, board, glass, wood, paper, textile, concrete, rock, marble, gypsum and ceramic materials which optionally may be coated, e.g., with paint, enamel and the like. The hard surface can also be a process equipment, e.g., a cooling tower, an osmotic membrane, a water treatment plant, a dairy, a food processing plant, a chemical or pharmaceutical process plant. Accordingly, the composition according to the present invention is useful in a conventional cleaning-in-place (C-I-P) system.

Enhancing agent

The present invention relates to enhancing agents selected from the group consisting of:

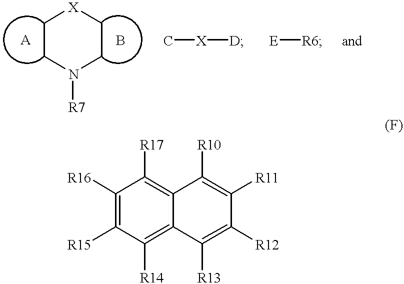

in which C, D, and E independently of each other are:

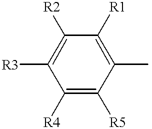

and R1, R2, R3, R4, R5, R6, R7, R10, R11, R12, R13, R14, R15, R16, R17 independently of each other are H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl; and X is a single bond or NH, NCH₃, NC₂H₅, O, S, N═N, CH═N, or CH═CH. In a preferred embodiment R1, R2, R3, R4, R5, R6, R7, R10, R11, R12, R13, R14, R15, R16, R17 independently of each other are H, OH, C₁₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl; and X is a single bond or NH, NCH₃, NC₂H₅, O, S, N═N, CH═N, or CH═CH.

A and B are six membered aromatic rings, and may independently of each other be substituted with H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl. In a preferred embodiment A and B may independently of each other be substituted with H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

One or more carbon atoms of the aromatic rings of A, B, C, D, E, and F may independently of each other be substituted with N or S, thus rendering said aromatic ring a heterocyclic ring.

In an embodiment X is a single bond or NH, NCH₃, NC₂H₅, O, S, N═N, CH═N, or CH═CH; preferably X is a single bond or NH, NCH₃, NC₂H₅, O, N═N, CH═N, or CH═CH; preferably X is a single bond or NH, NCH₃, NC₂H₅, S, N═N, CH═N, or CH═CH; more preferably X is a single bond or NH, NCH₃, NC₂H₅, N═N, CH═N, or CH═CH.

In an embodiment the substituents of A are independently of each other H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl. Preferably the substituents of A are independently of each other H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

In an embodiment the substituents of B are independently of each other H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl. Preferably the substituents of B are independently of each other H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

In an embodiment R1, R2, R3, R4, and R5 of C are independently of each other H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl. Preferably R1, R2, R3, R4, and R5 of C are independently of each other H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

In an embodiment R1, R2, R3, R4, and R5 of D are independently of each other H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, CO₁₋₄-alkyl or acyl. Preferably R1, R2, R3, R4, and R5 of D are independently of each other H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

In an embodiment R1, R2, R3, R4, and R5 of E are independently of each other H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl. Preferably R1, R2, R3, R4, and R5 of E are independently of each other H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H. C₁₋₄-alkyl or acyl.

In an embodiment R10, R11, R12, R13, R14, R15, R16 and R17 of F are independently of each other H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl. Preferably R10, R11, R12, R13, R14, R15, R16 and R17 of F are independently of each other H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

The terms “C₁₋ₙ-alkyl” wherein n can be from 1 through 8, as used herein, represent a branched or straight, saturated or unsaturated alkyl group having from one to the specified number of carbon atoms. Typical C₁₋₆-alkyl groups include, but are not limited to, methyl, ethyl, ethenyl (vinyl), n-propyl, iso-propyl, propenyl, isopropenyl, butyl, iso-butyl, sec-butyl, tert-butyl, crotyl, methallyl, pentyl, iso-pentyl, prenyl, hexyl, iso-hexyl and the like.

The term “acyl” as used herein refers to a monovalent substituent comprising a C₁₋₆-alkyl group linked through a carbonyl group; such as e.g. acetyl, propionyl, butyryl, isobutyryl, pivaloyl, valeryl, and the like.

In an embodiment at least one of the substituents of A are H, preferably at least two of the substituents of A are H, more preferably at least three of the substituents of A are H, most preferably at least four of the substituents of A are H, in particular all the substituents of A are H.

In another embodiment at least one of the substituents of B are H, preferably at least two of the substituents of B are H, more preferably at least three of the substituents of B are H, most preferably at least four of the substituents of B are H, in particular all the substituents of B are H.

In another embodiment at least one of the substituents R1, R2, R3, R4, and R5 of C are H, preferably at least two of the substituents R1, R2, R3, R4, and R5 of C are H, more preferably at least three of the substituents R1, R2, R3, R4, and R5 of C are H, most preferably at least four of the substituents R1, R2, R3, R4, and R5 of C are H, in particular all the substituents R1, R2, R3, R4, and R5 of C are H.

In another embodiment at least one of the substituents R1, R2, R3, R4, and R5 of D are H, preferably at least two of the substituents R1, R2, R3, R4, and R5 of D are H, more preferably at least three of the substituents R1, R2, R3, R4, and R5 of D are H, most preferably at least four of the substituents R1, R2, R3, R4, and R5 of D are H, in particular all the substituents R1, R2, R3, R4, and R5 of D are H.

In another embodiment at least one of the substituents R1, R2, R3, R4, and R5 of E are H, preferably at least two of the substituents R1, R2, R3, R4, and R5 of E are H, more preferably at least three of the substituents R1, R2, R3, R4, and R5 of E are H, most preferably at least four of the substituents R1, R2, R3, R4, and R5 of E are H, in particular all the substituents R1, R2, R3, R4, and R5 of E are H.

In another embodiment at least one of the substituents R10, R11, R12, R13, R14, R15, R16, and R17 of F are H, preferably at least two of the substituents R10, R11, R12, R13, R14, R15, R16, and R17 of F are H, more preferably at least three of the substituents R10, R11, R12, R13, R14, R15, R16, and R17 of F are H, more preferably at least four of the substituents R10, R11, R12, R13, R14, R15, R16, and R17 of F are H, more preferably at least five of the substituents R10, R11, R12, R13, R14, R15, R16, and R17 of F are H, more preferably at least seven of the substituents R10, R11, R12, R13, R14, R15, R16, and R17 of F are H, most preferably at least six of the substituents R10, R11, R12, R13, R14, R15, R16, and R17 of F are H, in particular all the substituents R10, R11, R12, R13, R14, R15, R16, and R17 of F are H.

In particular embodiments according to the invention the enhancing agent is selected from the group consisting of:

4-aminophenol;

p-Coumaric acid;

4,4′-Biphenol;

3,3′,5,5′-Tetramethylbenzidine;

4,4′-Diaminodiphenylamine sulfate;

4,4′-Dimethoxy-N-methyl-diphenylamine;

4,4′-Dihydroxydiphenyl ether;

4-Hydroxy-4′-dimethylamino azobenzene;

N′-Benzylidene-N,N-dimethyl-p-phenylenediamine;

4-Amino-4′-hydroxystilbene;

4′-Bromo-4-(dimethylamino)-chalcone;

3-Dimethylamino-9-ethylcarbazole;

Harmine, HCl H₂O;

Methylsyringate;

Propyl sinapate;

1,5-Diaminonaphthalene;

1,5-Dihydroxynaphthalene;

2,7-Dihydroxynaphthalene;

7-Methoxy-2-naphthol;

2-Hydroxy-1-naphthaldehyde;

2-Hydroxy-1-naphthoic acid;

8-Aminoquinoline;

10-Methylphenoxazine; and

3,7-Dibromophenoxazine-10-propionic acid.

The enhancing agent of the invention may be present in concentrations of from 1 to 1000 μM, preferably of from 5 to 500 μM, and more preferably from 10 to 200 μM.

Hydrogen peroxide/Oxygen

If the phenol oxidizing enzyme requires a source of hydrogen peroxide, the source may be hydrogen peroxide or a hydrogen peroxide precursor for in situ production of hydrogen peroxide, e.g., percarbonate or perborate, or a hydrogen peroxide generating enzyme system, (e.g., an oxidase together with a substrate for the oxidase, e.g., an amino acid oxidase together with a suitable amino acid), or a peroxycarboxylic acid or a salt thereof. Hydrogen peroxide may be added at the beginning of or during the process, e.g., typically in an amount corresponding to levels of from 0.001-25 mM, preferably to levels of from 0.005-5 mM, and particularly to levels of from 0.01-1 mM.

If the phenol oxidizing enzyme requires molecular oxygen, molecular oxygen from the atmosphere will usually be present in sufficient quantity. If more O₂ is needed, additional oxygen may be added.

Phenol Oxidizing Enzyme

In the context of the present invention the enzyme of the phenol oxidizing enzyme may be an enzyme possessing peroxidase activity or a laccase or a laccase related enzyme.

The enzyme of the invention may typically be present in concentrations of from 1 to 100000 μg enzyme protein per liter aqueous solution, preferably of from 5 to 50000 μg enzyme protein per liter aqueous solution, more preferably of from 10 to 10000 μg enzyme protein per liter aqueous solution, and most preferably of from 50 to 5000 μg enzyme protein per liter aqueous solution.

Peroxidases and Compounds possessing Peroxidase Activity

Compounds possessing peroxidase activity may be any peroxidase enzyme comprised by the enzyme classification (EC 1.11.1.7), or any fragment derived therefrom, exhibiting peroxidase activity.

Preferably, the peroxidase according to the invention is producible by plants (e.g. horseradish or soybean peroxidase) or microorganisms such as fungi or bacteria.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucaria (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g., Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g., T. versicolor (e.g. PR4 28-A).

Further preferred fungi include strains belonging to the subdivision Zygomycotina, class Mycoraceae, e.g., Rhizopus or Mucor, in particular Mucor hiemalis.

Some preferred bacteria include strains of the order Actinomycetales, e.g. Streptomyces spheroides (ATTC 23965), Streptomyces thermoviolaceus (IFO 12382) or Streptoverticillum verticillium ssp. verticillium.

Other preferred bacteria include Rhodobacter sphaeroides, Rhodomonas palustri, Streptococcus lactis, Pseudomonas purrocinia (ATCC 15958), Pseudomonas fluorescens (NRRL B-11) and Bacillus strains, e.g. Bacillus pumilus (ATCC 12905) and Bacillus stearothermophilus.

Further preferred bacteria include strains belonging to Myxococcus, e.g., M. virescens.

The peroxidase may furthermore be one which is producible by a method comprising cultivating a host cell transformed with a recombinant DNA vector which carries a DNA sequence encoding said peroxidase as well as DNA sequences encoding functions permitting the expression of the DNA sequence encoding the peroxidase, in a culture medium under conditions permitting the expression of the peroxidase and recovering the peroxidase from the culture.

Particularly, a recombinantly produced peroxidase is a peroxidase derived from a Coprinus sp., in particular C. macrorhizus or C. cinereus according to WO 92/16634.

In the context of this invention, compounds possessing peroxidase activity comprise peroxidase enzymes and peroxidase active fragments derived from cytochromes, haemoglobin or peroxidase enzymes.

Determination of Peroxidase Activity (POXU)

One peroxidase unit (POXU) is the amount of enzyme which under the following conditions catalyze the conversion of 1 μmole hydrogen peroxide per minute:

0.1 M phosphate buffer pH 7.0

0.88 mM hydrogen peroxide

1.67 mM 2,2′-azino-bis(3-ethylbenzothiazoline-6-sulfonate) (ABTS)

30° C.

The reaction is followed for 60 seconds (15 seconds after mixing) by the change in absorbance at 418 nm, which should be in the range 0.15 to 0.30.

For calculation of activity is used an absorption coefficient of oxidized ABTS of 36 mM⁻¹ cm⁻¹ and a stoichiometry of one μmole H₂O₂ converted per two μmole ABTS oxidized.

Laccases and Laccase Related Enzymes

In the context of this invention, laccases and laccase related enzymes comprise any laccase enzyme comprised by the enzyme classification (EC 1.10.3.2), any catechol oxidase enzyme comprised by the enzyme classification (EC 1.10.3.1), any bilirubin oxidase enzyme comprised by the enzyme classification (EC 1.3.3.5) or any monophenol monooxygenase enzyme comprised by the enzyme classification (EC 1.14.18.1).

The above-mentioned enzymes may be microbial, i.e. derived from bacteria or fungi (including filamentous fungi and yeasts), or they may be derived from plants.

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinus, e.g., C. cinereus, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Pycnoporus, e.g. P. cinnabarinus, Phlebia, e.g., P. radita (WO 92/01046), or Coriolus, e.g., C. hirsutus (JP 2-238885).

Suitable examples from bacteria include a laccase derivable from a strain of Bacillus.

A laccase derived from Coprinus, Myceliophthora, Polyporus, Pycnoporus, Scytalidium or Rhizoctonia is preferred; in particular a laccase derived from Coprinus cinereus, Myceliophthora thermophila, Polyporus pinsitus, Pycnoporus cinnabarinus, Scytalidium thermophilum or Rhizoctonia solani.

The laccase or the laccase related enzyme may furthermore be one which is producible by a method comprising cultivating a host cell transformed with a recombinant DNA vector which carries a DNA sequence encoding said laccase as well as DNA sequences encoding functions permitting the expression of the DNA sequence encoding the laccase, in a culture medium under conditions permitting the expression of the laccase enzyme, and recovering the laccase from the culture.

Determination of Laccase Activity (LACU)

Laccase activity is determined from the oxidation of syringaldazin under aerobic conditions. The violet colour produced is photometered at 530 nm. The analytical conditions are 19 mM syringaldazin, 23 mM acetate buffer, pH 5.5, 30° C., 1 min. reaction time.

1 laccase unit (LACU) is the amount of enzyme that catalyses the conversion of 1.0 μmole syringaldazin per minute at these conditions.

Determination of Laccase Activity (LAMU)

Laccase activity is determined from the oxidation of syringaldazin under aerobic conditions. The violet colour produced is photometered at 530 nm. The analytical conditions are 19 mM syringaldazin, 23 mM Tris/maleate buffer, pH 7.5, 30° C., 1 min. reaction time.

1 laccase unit (LAMU) is the amount of enzyme that catalyses the conversion of 1.0 μmole syringaldazin per minute at these conditions.

The composition

The present invention provides an enzymatic antimicrobial composition comprising a phenol oxidizing enzyme system and an enhancing agent of a formula selected from group consisting of:

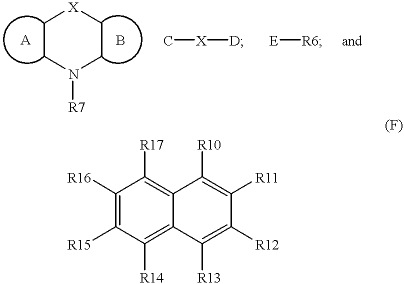

as described above.

The antimicrobial composition according to the invention may be formulated as a solid or a liquid.

When formulated as a liquid, the composition is typically an aqueous composition.

When formulated as a solid, the composition is typically a powder, a granulate, a paste or a gelled product.

It is preferred to use a two-part formulation system in the cases where hydrogen peroxide is needed, whereby the hydrogen peroxide is separate from the other components.

The composition of the invention may further comprise auxiliary agents such as wetting agents, thickening agents, buffer, stabilisers, perfume, colorants, fillers and the like.

Useful wetting agents are surfactants, i.e., non-ionic, anionic, amphoteric or zwitterionic surfactants.

The composition of the invention may be a concentrated product or a ready-to-use product. In use, the concentrated product is typically diluted with water to provide a medium having an effective antimicrobial activity, applied to the object to be disinfected or preserved, and allowed to react with the microorganisms present.

The optimum pH of such an aqueous composition is usually a compromise between the optimum stability and optimum activity of the enzyme in question. In one aspect of the invention pH is in the range of pH 3 to 10.5 (such as pH 4 to 6 or pH 8 to 10), and in another aspect of the invention pH is in the range of pH 4 to 10, preferably pH 5 to 9, and more preferably pH 6 to 8.

In an embodiment, the present invention also provides a medical catheter comprising the composition of the invention.

The method

The present invention also provides a method for killing or inhibiting microbial cells comprising treating said microbial cells with the composition of the invention. Said treatment may be carried out with an effective amount of said composition.

As an “effective amount” is meant an amount suitable for obtaining the required antimicrobial effect in the chosen application; e.g. to reduce the number of living cells to 10%, 1% or less than 1%; or to prevent the number of living cells from doubling during 12 hours, 1 day, 5 days, 30 days or more than 30 days.

The composition of the invention may be capable of reducing the number of living cells (killing) of E. coli (DSM1576) to less than 50% (preferably less than 75%, more preferably less than 90%, most preferably less than 95%, in particular at least 99%), when incubated 10 min. at 20° C. in an aqueous solution containing 1 mg/L of the composition.

The composition may also be capable of increasing the time before outgrowth (inhibition) of E. coli (DSM1576) at 25° C. in a microbial growth substrate containing 1 mg/L of the composition by at least 5%, preferably at least 10%, more preferably at least 25%, most preferably at least 50%, and in particular at least 100%.

Uses

The composition of the invention may be incorporated into a detergent or cleaning composition typically comprising other enzyme types as well (see below).

The composition of the invention can also be used for inhibiting microorganisms present in laundry, by treating the laundry with a soaking, washing or rinsing liquor comprising an effective amount of the composition.

When used for preservation of paint, food, beverages, cosmetics such as lotions (e.g. eye lotions), liquids, creams, gels, pastes, ointments (e.g. eye ointments), soaps, shampoos, conditioners, antiperspirants, deodorants, mouth wash, nasal sprays, contact lens products, enzyme formulations, or food ingredients, the composition used in the method of the present invention may be incorporated into e.g. water based paint, unpreserved food, beverages, cosmetics, contact lens products, food ingredients or anti-inflammatory product in an amount effective for killing or inhibiting growth of microbial cells.

In particular, the composition of the invention may be used as a preservation agent or a disinfection agent in water based paints (see below).

Furthermore, the composition according to the present invention may by useful as a disinfectant, e.g., in the treatment of acne, infections in the eye or the mouth, skin infections; in antiperspirants or deodorants; in foot bath salts; for cleaning and disinfection of contact lenses, hard surfaces, teeth (oral care), wounds, bruises and the like.

In general the composition of the present invention is useful for cleaning, disinfecting or inhibiting microbial growth on any hard surface. Examples of surfaces, which may advantageously be contacted with the composition of the invention are surfaces of process equipment used, e.g., in dairies, chemical or pharmaceutical process plants, water sanitation systems, paper pulp processing plants, water treatment plants, and cooling towers. The composition of the invention may be used in an amount, which is effective for cleaning, disinfecting or inhibiting microbial growth on the surface in question.

In particular, the composition of the invention may be used for disinfecting and inhibiting microbial growth in paper and pulp processing plants.

Further, it is contemplated that the composition of the invention can advantageously be used in a cleaning-in-place (C.I.P.) system for cleaning of process equipment of any kind.

The method of the invention may additionally be used for cleaning surfaces and cooking utensils in food processing plants and in any area in which food is prepared or served such as hospitals, nursing homes, restaurants, especially fast food restaurants, delicatessens and the like. It may also be used as an antimicrobial in food products and would be especially useful as a surface antimicrobial for cheese, fruits and vegetables and for food in salad bars.

The composition of the present invention is also useful for microbial control of water lines, and for disinfection of water, in particular for disinfection of industrial water.

In an embodiment, the composition of the present invention is also useful for treating medical catheters to kill or inhibit microbes present on the surface of said medical catheters. The composition of the invention can also be used for treating wounds.

Conservation/preservation of paints

Conservation of paint products in cans has in the art been accomplished by adding non-enzymatic organic biocides to the paints. In the context of the invention paint is construed as a substance comprising a solid coloring matter dissolved or dispersed in a liquid vehicle such as water, organic solvent and/or oils, which when spread over a surface, dries to leave a thin colored, decorative and/or protective coating. Typically isothiazoliones, such as 5-chlor-2-methyl-4-thia-zoli-3-on, has been added to the paint as biocides to inhibit/prevent microbial growth in the paint. The method of the invention can however suitably be applied in this field, thereby solving the problem of the ever present environmental bio-hazards of using toxic organic biocides by replacing these toxic biocides with environmentally compatible enzymes. Thus the present invention provides a method for conservation of a paint comprising contacting said paint with a phenol oxidizing enzyme and an enhancing agent according to the invention. Further the invention provides a paint composition comprising a phenol oxidizing enzyme and an enhancing agent as described in the present invention.

The paint is preferably a water based paint, i.e. the solids of the paint is dispersed in an aqueous solution. The paint may contain 0-20 % organic solvent, preferable 0-10%, e.g. 0-5%.

The enzyme may be added to the paint in an amount of 0.0001-100 mg active enzyme protein per liter paint, preferably 0.001-10 mg/l, e.g. 0.01-5 mg/l, while the enhancing agent may be added in an amount of 10-500 μM, preferably 25-250 μM, e.g. 100 μM of the paint composition.

Detergent Compositions

The antimicrobial composition of the invention may be added to and thus become a component of a detergent composition.

The detergent composition of the invention may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the invention provides a detergent additive comprising the antimicrobial composition of the invention. The detergent additive as well as the detergent composition may comprise one or more other enzymes such as a protease, a lipase, a cutinase, an amylase, a carbohydrase, a cellulase, a pectinase, a mannanase, an arabinase, a galactanase, a xylanase, an oxidase, e.g., a laccase, and/or a peroxidase.

In general the properties of the chosen enzyme(s) should be compatible with the selected detergent, (i.e. pH-optimum, compatibility with other enzymatic and non-enzymatic ingredients, etc.), and the enzyme(s) should be present in effective amounts. Proteases: Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include Alcalase™, Savinase™, Primase™, Duralase™, Esperase™, and Kannase™ (Novozymes A/S), Maxatase™, Maxacal™, Maxapem™, Properase™, Purafect™, Purafect OXP™, FN2™, and FN3™ (Genencor International Inc.).

Lipases: Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include Lipolase™ and Lipolase Ultra™ (Novozymes A/S).

Amylases. Suitable amylases (α and/or β) include those of bacterial or fungal origin. Chemically modified or protein engineered mutants are included. Amylases include, for example, α-amylases obtained from Bacillus, e.g. a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramyl™, Termamyl™, Fungamyl™ and BAN™ (Novozymes A/S), Rapidase ™ and Purastar™ (from Genencor International Inc.).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novozymes A/S), Clazinase™, and Puradax HA™ (Genencor International Inc.), and KAC-500(B)™ (Kao Corporation).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO 93/24618, WO 95/10602, and WO 98/15257.

The detergent enzyme(s) may be included in a detergent composition by adding separate additives containing one or more enzymes, or by adding a combined additive comprising all of these enzymes. A detergent additive of the invention, i.e. a separate additive or a combined additive, can be formulated e.g. as a granulate, a liquid, a slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme Ifs preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent composition of the invention may be in any convenient form, e.g., a bar, a tablet, a powder, a granule, a paste or a liquid. A liquid detergent may be aqueous, typically containing up to 70% water and 0-30% organic solvent, or non-aqueous.

The detergent composition comprises one or more surfactants, which may be non-ionic including semi-polar and/or anionic and/or cationic and/or zwitterionic. The surfactants are typically present at a level of from 0.1 % to 60% by weight.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

When included therein the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The detergent may contain 0-65% of a detergent builder or complexing agent such as zeolite, diphosphate, triphosphate, phosphonate, carbonate, citrate, nitrilotriacetic acid, ethylenediaminetetraacetic acid, diethylenetriaminepentaacetic acid, alkyl- or alkenylsuccinic acid, soluble silicates or layered silicates (e.g. SKS-6 from Hoechst).

The detergent may comprise one or more polymers. Examples are carboxymethylcellulose, poly(vinylpyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid copolymers.

The detergent may contain a bleaching system, which may comprise a H₂O₂ source such as perborate or percarbonate, which may be combined with a peracid-forming bleach activator such as tetraacetylethylenediamine or nonanoyloxybenzenesulfonate. Alternatively, the bleaching system may comprise peroxyacids of e.g. the amide, imide, or sulfone type.

The enzyme(s) of the detergent composition of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in e.g. WO 92/19709 and WO 92/19708.

The detergent may also contain other conventional detergent ingredients such as e.g. fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

The present invention is further illustrated in the following examples, which are not in any way intended to limit the scope of the invention as claimed.

### EXAMPLE 1

### Antimicrobial Activity of Enhancing Agents

Screening for enhancing agents with antimicrobial activity was performed in 384 well microtiter plates by measuring the ability of laccase/enhancing agent combinations to inhibit growth of Pseudomonas putida ATCC12633.

Enhancing agents were dissolved in EtOH at a concentration of 10 mM and stored at 4° C. From these stock solutions a 96 well master plate containing ˜200 μM enhancing agent was generated by adding 6 μl of enhancing agent stock into 280 μl milliQ water. The layout of the master plate was as shown in Table 1.

TABLE 1
Layout of master plate containing enhancing agent. Columns 1 and 2 contain
50 mM HEPES buffer pH 7.0 and m1-m40 are numbered enhancing agents.

| | 1 | 2 | 3 | 4 | 5 | 6 | 7 | 8 | 9 | 10 | 11 | 12 |
|---|---|---|---|---|---|---|---|---|---|---|---|---|
| A | Hepes | Hepes | m1 | m1 | m9 | M9 | m17 | m17 | m25 | m25 | m33 | m33 |
| B | Hepes | Hepes | m2 | m2 | m10 | M10 | m18 | m18 | m26 | m26 | m34 | m34 |
| C | Hepes | Hepes | m3 | m3 | m11 | M11 | m19 | m19 | m27 | m27 | m35 | m35 |
| D | Hepes | Hepes | m4 | m4 | m12 | M12 | m20 | m20 | m28 | m28 | m36 | m36 |
| E | Hepes | Hepes | m5 | m5 | m13 | M13 | m21 | m21 | m29 | m29 | m37 | m37 |
| F | Hepes | Hepes | m6 | m6 | m14 | M14 | m22 | m22 | m30 | m30 | m38 | m38 |
| G | Hepes | Hepes | m7 | m7 | m15 | M15 | m23 | m23 | m31 | m31 | m39 | m39 |
| H | Hepes | Hepes | m8 | m8 | m16 | M16 | m24 | m24 | m32 | m32 | m40 | m40 |

Pseudomonas putida ATCC12633 was grown in a dilution series over night in LB medium and a late exponential culture was harvested by centrifugation (5 min @ 4000 RPM in a Microcentrifuge 154, Ole Dich, Denmark) and washed twice in 50 mM HEPES buffer pH 7.0. OD@490 was adjusted to 0.05 corresponding to approximately 10E5 cells/ml.

TABLE 2
Layout of screenings plate. Only half of the 384 well plate is shown. Rows 3
5, 7, 9, 11 contain enhancing agent only, whereas rows 4, 6, 8, 10, 12 contain
enhancing agent and laccase. Rows 1 and 2 are negative controls as well as a dilution
series of cells are included in order to evaluate the amount of killing observed by the
laccase / enhancing agent system. All assays were run in duplicate.

| | 1 | 2 | 3 | 4 | 5 | 6 | 7 | 8 | 9 | 10 | 11 | 12 |
|---|---|---|---|---|---|---|---|---|---|---|---|---|
| A | Hepes | Hepes | m1 | m1 | m9 | m9 | m17 | m17 | m25 | m25 | m33 | m33 |
| B | Hepes | Hepes | m1 | m1 | m9 | m9 | m17 | m17 | m25 | m25 | m33 | m33 |
| C | 10E0 | 10E0 | m2 | m2 | m10 | m10 | m18 | m18 | m26 | m26 | m34 | m34 |
| D | 10E0 | 10E0 | m2 | m2 | m10 | m10 | m18 | m18 | m26 | m26 | m34 | m34 |
| E | 10E1 | 10E1 | m3 | m3 | m11 | m11 | m19 | m19 | m27 | m27 | m35 | m35 |
| F | 10E1 | 10E1 | m3 | m3 | m11 | m11 | m19 | m19 | m27 | m27 | m35 | m35 |
| G | 10E2 | 10E2 | m4 | m4 | m12 | m12 | m20 | m20 | m28 | m28 | m36 | m36 |
| H | 10E2 | 10E2 | m4 | m4 | m12 | m12 | m20 | m20 | m28 | m28 | m36 | m36 |
| I | 10E3 | 10E3 | m5 | m5 | m13 | m13 | m21 | m21 | m29 | m29 | m37 | m37 |
| J | 10E3 | 10E3 | m5 | m5 | m13 | m13 | m21 | m21 | m29 | m29 | m37 | m37 |
| K | Hepes | Hepes | m6 | m6 | m14 | m14 | m22 | m22 | m30 | m30 | m38 | m38 |
| L | Hepes | Hepes | m6 | m6 | m14 | m14 | m22 | m22 | m30 | m30 | m38 | m38 |
| M | LB | LB | m7 | m7 | m15 | m15 | m23 | m23 | m31 | m31 | m39 | m39 |
| N | LB | LB | m7 | m7 | m15 | m15 | m23 | m23 | m31 | m31 | m39 | m39 |
| O | | | m8 | m8 | m16 | m16 | m24 | m24 | m32 | m32 | m40 | m40 |
| P | | | m8 | m8 | m16 | m16 | m24 | m24 | m32 | m32 | m40 | m40 |

10 μl of the enhancing agent from the 96 well master plate (Table 1) was transferred to a 384 well plate in duplicate as shown in table 2. The 384 well screening plate was pre-incubated at 40° C. on a heat block (Techne DB-2A, Buch & Holm A/S, Denmark) before the assay was started by addition of 35 μl of preheated buffer (40° C.) containing cells (10E5 CFU/ml) with or without laccase (12,5 μg/ml). The reaction was incubated for 25 min at 40° C., and 45 μl LB medium was added to allow cells to grow. Growth at 30° C. was monitored using a Polarstar Galaxy microtitter plate reader (BMG Labtechnologies, Germany) in the absorbance mode. The development in absorbance was measured online for 16 hours at 480 nm, and the resulting growth curves were used for evaluation of the anti-microbial activity of the different laccase/enhancing agent combinations. The reduction in colony forming units was estimated by comparison to the standard curve, and anti-microbial enhancing agents were categorized in two groups: Group 1 compounds showed between 10E2 and 10E5 reduction and group 2 showed more than 10E5 reduction (i.e. total kill). Results are shown in tables 3-6.

TABLE 3
Enhancing agents with antimicrobial activity mediated by MtL:

| | | | | CFU |
|---|---|---|---|---|
| CAS no. | Name | | Supplier | reduction |
| 123-30-8 | 4-aminophenol | 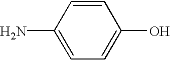 | Aldrich | >10⁵ |
| 54827-17-7 | 3,3′,5,5′- Tetramethylbenzidine | 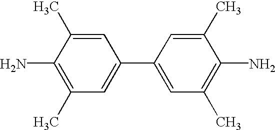 | Acros | 10²-10⁵ |
| 27151-57-1 | 4,4′-Dimethoxy-N- methyldiphenylamine | 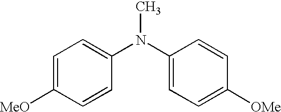 | Lancaster | 10²-10⁵ |
| 1965-09-9 | 4,4′- Dihydroxyphenyl ether | 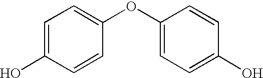 | Sigma- Aldrich (Acros) | >10⁵ |
| 2496-15-3 | 4-Hydroxy-4′- dimethylamino azobenzene | 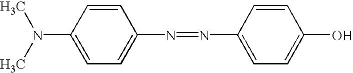 | TCl | >10⁵ |
| | 3-Dimethylamino-9- ethylcarbazole | 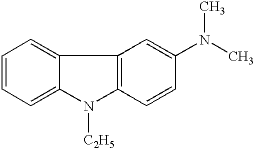 | Novozymes | >10⁵ |
| 884-35-5 | Methylsyringate | 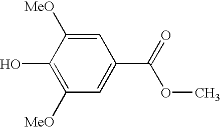 | Lancaster | 10²-10⁵ |
| 2243-62-1 | 1,5- Diaminonaphthalene | 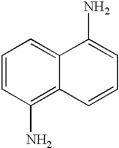 | Novozymes | >10⁵ |
| 582-17-2 | 2,7- Dihydroxynaphthalene | 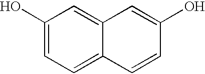 | Novozymes | 10²-10⁵ |
| 25782-99-4 | 10- Methylphenoxazine | 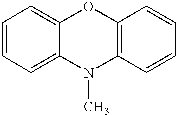 | Novozymes | 10²-10⁵ |

TABLE 4
Enhancing agents with antimicrobial activity mediated by RsL:

| | | | | CFU |
|---|---|---|---|---|
| CAS no. | Name | | Supplier | reduction |
| 123-30-8 | 4-aminophenol | 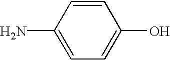 | Aldrich | >10⁵ |
| 92-88-6 | 4,4′-Biphenol, 97% | 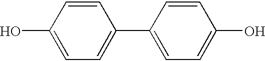 | Aldrich | 10²-10⁵ |
| 6369-04-6 | 4,4′- Diaminophenylam- ine sulfate, tech. 85% | 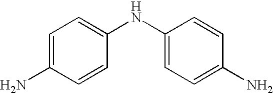 | Janssen Chemica | >10⁵ |
| 1965-09-9 | 4,4′- Dihydroxyphenyl ether | 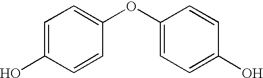 | Sigma- Aldrich (Acros) | >10⁵ |
| 2496-15-3 | 4-Hydroxy-4′- dimethylamino azobenzene | 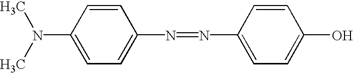 | TCl | >10⁵ |
| | 32-Dimethylamino-9- ethylcarbazole | 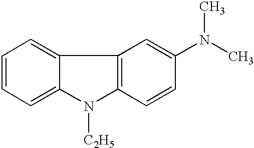 | Novozymes | >10⁵ |
| 884-35-5 | Methylsyringate | 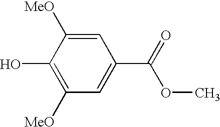 | Lancaster | 10²-10⁵ |
| | Propyl sinapate | 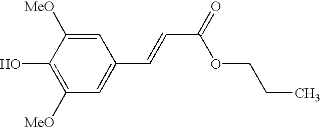 | Novozymes | >10⁵ |
| 2243-62-1 | 1,5- Diaminonaphthalene | 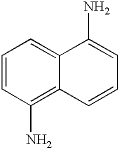 | Novozymes | >10⁵ |
| 83-56-7 | 1,5- Dihydroxynaphthalene | 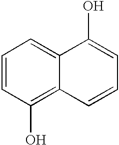 | Novozymes | 10²-10⁵ |
| 582-17-2 | 2,7- Dihydroxynaphthalene | 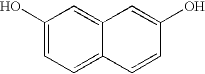 | Novozymes | 10²-10⁵ |
| 2283-08-1 | 2-Hydroxy-1- naphthoic acid | 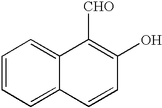 | TCl | >10⁵ |
| 578-66-5 | 8-Aminoquinoline | 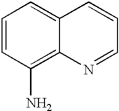 | Aldrich | >10⁵ |
| | 3,7- Dibromophenoxazine- 10-propionic acid | 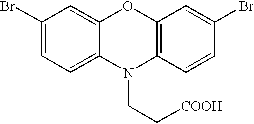 | Novozymes | >10⁵ |

TABLE 5
Enhancing agents with anti-microbial activity mediated by CcL:

| | | | | CFU |
|---|---|---|---|---|
| CAS no. | Name | | Supplier | reduction |
| 123-30-8 | 4-aminophenol | 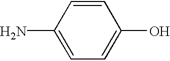 | Aldrich | >10⁵ |
| 92-88-6 | 4,4′-Biphenol, 97% | 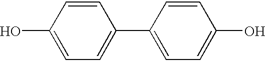 | Aldrich | 10²-10⁵ |
| 54827-17-7 | 3,3′,5,5′- Tetramethylbenzidine | 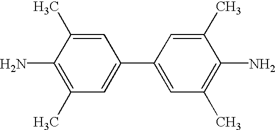 | Acros | 10²-10⁵ |
| 6369-04-6 | 4,4′- Diaminodiphenylam- ine sulfate, tech. 85% | 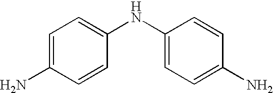 | Janssen Chemica | >10⁵ |
| 27151-57-1 | 4,4′-Dimethoxy-N- methyldiphenylamine | 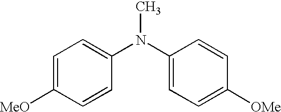 | Lancaster | 10²-10⁵ |
| 1965-09-9 | 4,4′- Dihydroxydiphenyl ether | 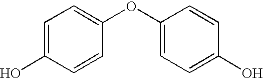 | Sigma- Aldrich (Acros) | >10⁵ |
| 2496-15-3 | 4-Hydroxy-4′- dimethylamino azobenzene | 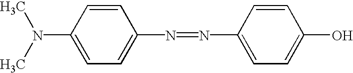 | TCl | >10⁵ |
| 889-37-2 | N′-Benzylidene- N,N-dimethyl-p- phenylenediamine | 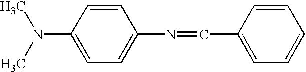 | Sigma- Aldrich | 10²-10⁵ |
| | 3-Dimethylamino-9- ethylcarbazole | 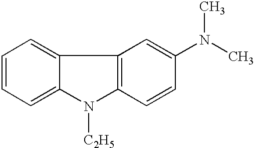 | Novozymes | >10⁵ |
| 884-35-5 | Methylsyringate | 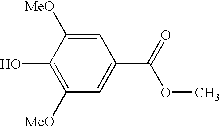 | Lancaster | 10²-10⁵ |
| | Propyl sinapate | 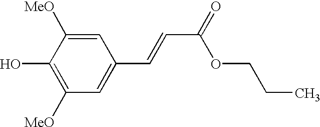 | Novozymes | >10⁵ |
| 2243-62-1 | 1,5- Diaminonaphthalene | 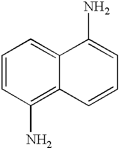 | Novozymes | >10⁵ |
| 83-56-7 | 1,5- Dihydroxynaphthalene | 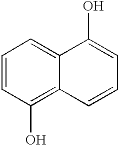 | Novozymes | 10²-10⁵ |
| 582-17-2 | 2,7- Dihydroxynaphthalene | 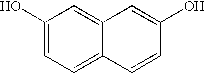 | Novozymes | 10²-10⁵ |
| 5060-82-2 | 7-Methoxy-2- naphthol, 98% | 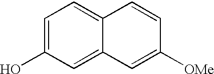 | Aldrich | 10²-10⁵ |
| 2283-08-1 | 2-Hydroxy-1- naphthoic acid | 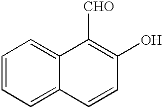 | TCl | >10⁵ |
| 578-66-5 | 8-Aminoquinoline | 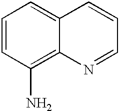 | Aldrich | >10⁵ |
| 25782-99-4 | 10- Methylphenoxazine | 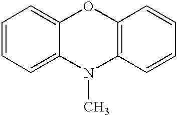 | Novozymes | 10²-10⁵ |
| | 3,7- Dibromophenoxazine- 10-propionic acid | 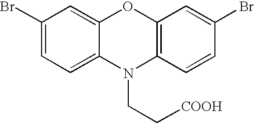 | Novozymes | >10⁵ |
| | 3-(3- Hydroxyphenyl)rhod- anine | 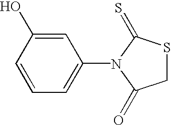 | Sima- Aldrich | 10²-10⁵ |

TABLE 6
Enhancing agents with anti-microbial activity mediated by PpL:

| | | | | CFU |
|---|---|---|---|---|
| CAS no. | Name | | Supplier | reduction |
| 123-30-8 | 4-aminophenol | 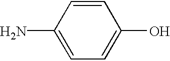 | Aldrich | >10⁵ |
| 92-88-6 | 4,4′-Biphenol, 97% | 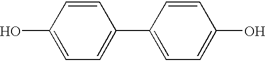 | Aldrich | 10²-10⁵ |
| 54827-17-7 | 3,3′,5,5′- Tetramethylbenzidine | 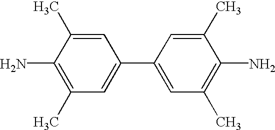 | Acros | 10²-10⁵ |
| 6369-04-6 | 4,4′- Diaminophenylam- ine sulfate, tech. 85% | 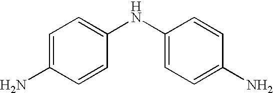 | Janssen Chemica | >10⁵ |
| 27151-57-1 | 4,4′-Dimethoxy-N- methyldiphenylamine | 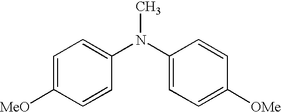 | Lancaster | 10²-10⁵ |
| 1965-09-9 | 4,4′- Dihydroxyphenyl ether | 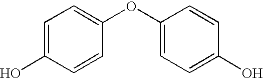 | Sigma- Aldrich (Acros) | >10⁵ |
| 2496-15-3 | 4-Hydroxy-4′- dimethylamino azobenzene | 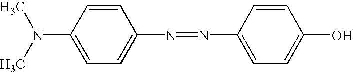 | TCl | >10⁵ |
| 889-37-2 | N′-Benzylidene- N,N-dimethyl-p- phenylenediamine | 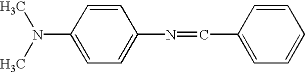 | Sigma- Aldrich | 10²-10⁵ |
| 836-44-2 | 4-Amino-4′- hydroxystilbene | 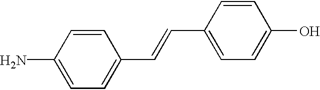 | Lancaster | 10²-10⁵ |
| | 3-Dimethylamino-9- ethylcarbazole | 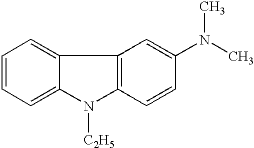 | Novozymes | >10⁵ |
| 884-35-5 | Methylsyringate | 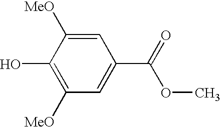 | Lancaster | 10²-10⁵ |
| 2243-62-1 | 1,5- Diaminonaphthalene | 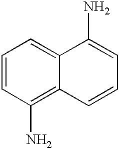 | Novozymes | >10⁵ |
| 83-56-7 | 1,5- Dihydroxynaphthalene | 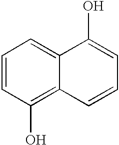 | Novozymes | 10²-10⁵ |
| 582-17-2 | 2,7- Dihydroxynaphthalene | 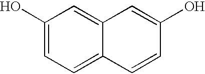 | Novozymes | 10²-10⁵ |
| 5060-82-2 | 7-Methyl-2- naphthol, 98% | 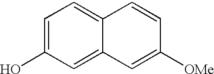 | Aldrich | 10²-10⁵ |
| 25782-99-4 | 10- Methylphenoxazine | 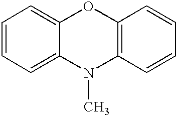 | Novozymes | 10²-10⁵ |
| | 3,7- Dibromophenoxazine- 10-propionic acid | 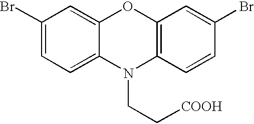 | Novozymes | 10²-10⁵ |
| | 3-(3- Hydroxyphenyl)rhod- anine | 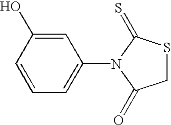 | Sima- Aldrich | 10²-10⁵ |
| | p-curmaric acid | 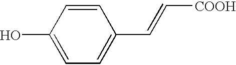 | Novozymes | 10²-10⁵ |

Reagents

50 mM HEPES buffer (Sigma Catalogue # H3375) pH 7.00

LB Bouillon was from Merck, #0285. 25 g mix was added to 1000 ml water and then autoclaved.

The laccases used are described in:

| | |
|---|---|
| Myceliophthora thermophila (MtL) | WO 95/33836 |
| Rhizoctonia solani (RsL) | WO 95/07988 |
| Coprinus cinereus (CcL) | WO 97/08325 |
| Polyporus pinsitus (PpL) | WO 96/00290 |

which are hereby incorporated by reference.

Microtiter plates (96 well and 384 well plates) were obtained from Nalge Nunc International (Denmark). Enhancing agents were supplied as indicated in Table 3-6.

### EXAMPLE 2

### Antimicrobial Activity of Mediators Oxidized by Peroxidose

Antibacterial activity of 4-aminophenol, 4-hydroxy-4′-dimethylamino azobenzene, 4,4′-biphenol, 10-methylphenoxazine and 4,4′-dihydroxydiphenyl ether was evaluated when oxidized by Coprinus cinereus peroxidase (rCiP) (available from Novozymes A/S).

Antimicrobial activity was determined on Pseudomonas putida (ATCC 12633), the cells were grown in Tryptone Soy Broth, TSB (Oxoid CM129) overnight, washed two times in sterile 0.9% saline and suspended in 50 mM HEPES buffer (Sigma H3375) at pH 7 to a cell concentration of approximately 10⁶ CFU/ml. The mediators were added to the cell suspension to a final concentration of 50 μM, rCiP was added to the concentration 4 POXU/L and the enzyme reaction was started by addition of hydrogen peroxide to a final concentration of 0.5 mM. Antimicrobial activity was determined after 20 minutes incubation at 40° C., by a 10-fold dilution of the cell suspension into TSB substrate and incubation overnight. Antimicrobial activity was determined as log₁₀ reduction, thus a log₁₀ reduction of 2 corresponds to a kill of 99%.

TABLE 7
Antimicrobial activity of mediators oxidized by peroxidase.

| Mediator | Log₁₀ reduction |
|---|---|
| 4-amino phenol | 2.5 |
| 4-hydroxy-4′-dimethylamino azobenzene | 6* |
| 4,4′biphenol | 1.5 |
| 10-methylphenoxazine | 3 |
| 4,4′-dihydroxydiphenyl ether | 2.5 |

*corresponds to a total kill

None of the evaluated mediators showed any antimicrobial activity without rCiP or when combined with hydrogen peroxide.

1. An enzymatic antimicrobial composition comprising a phenol oxidizing enzyme system and an enhancing agent of a formula selected from the group consisting of: 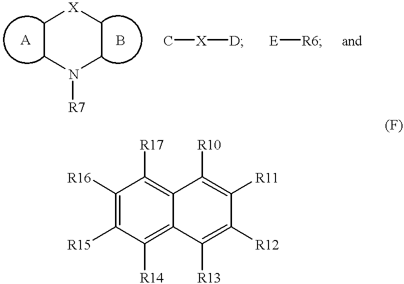
in which
C, D, and E independently of each other are: 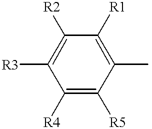
R1, R2, R3, R4, R5, R6, R7, R10, R11, R12, R13, R14, R15, R16, R17 independently of each other are H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl;
X is a single bond or NH, NCH₃, NC₂H₅, O, S, N═N, CH═N, or CH═CH; and
A and B are six membered aromatic rings, and are independently of each other be substituted with H, OH, C₁₋₈-alkyl, acyl, SO₃H, NO₂, CN, Cl, Br, F, NHR8, N(R8)₂, OR9, C₁₋₈-alkyl-OR9, or C₁₋₈-alkyl-OOR9; wherein R8 and R9 are H, C₁₋₄-alkyl or acyl.

2. The composition of claim 1, in which R1, R2, R3, R4, R5, R6, R7, R10, R11, R12, R13, R14, R15, R16, R17 independently of each other are H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl; and X is a single bond or NH, NCH₃, NC₂H₅, O, S, N═N, CH═N, or CH═CH; and A and B are independently of each other be substituted with H, OH, C₁₋₄-alkyl, acyl, NO₂, Cl, Br, NHR8, N(R8)₂, OR9, C₁₋₄-alkyl-OR9, or C₁₋₄-alkyl-OOR9; wherein R8 and R9 are H, C₁₋₄-alkyl or acyl.

3. The composition of claim 1, in which one or more carbon atoms of the aromatic rings of A, B, C, D, E, and F are substituted with N or S.

4. The composition of claim 1, in which at least one of R1, R2, R3, R4, and R5 is H.

5. The composition of claim 1, in which at least one of R10, R11, R12, R13, R14, R15, R16, and R17 is H.

6. The composition of claim 1, in which the enhancing agent is selected from the group consisting of:
4-aminophenol;
p-Coumaric acid;
4,4′-Biphenol;
3,3′,5,5′-Tetramethylbenzidine;
4,4′-Diaminodiphenylamine sulfate;
4,4′-Dimethoxy-N-methyl-diphenylamine;
4,4′-Dihydroxydiphenyl ether;
4-Hydroxy-4′-dimethylamino azobenzene;
N′-Benzylidene-N,N-dimethyl-p-phenylenediamine;
4-Amino-4′-hydroxystilbene;
4′-Bromo-4-(dimethylamino)-chalcone;
3-Dimethylamino-9-ethylcarbazole;
Harmine, HCl H₂O;
Methylsyringate;
Propyl sinapate;
1,5-Diaminonaphthalene;
1,5-Dihydroxynaphthalene;
2,7-Dihydroxynaphthalene;
7-Methoxy-2-naphthol;
2-Hydroxy-1-naphthaldehyde;
2-Hydroxy-1-naphthoic acid;
8-Aminoquinoline;
b 10-Methylphenoxazine; and
3,7-Dibromophenoxazine-10-propionic acid.

7. The composition of claim 1, in which the phenol oxidizing enzyme system is a peroxidase and a hydrogen peroxide source.

8. The composition of claim 7, wherein the peroxidase is horseradish peroxidase, soybean peroxidase or a peroxidase enzyme derived from Coprinus, Bacillus, or Myxococcus.

9. The composition of claim 8, wherein the peroxidase is derived from Coprinus cinereus or Coprinus macrorhizus.

10. The composition of claim 7, wherein the hydrogen peroxide source is hydrogen peroxide or a hydrogen peroxide precursor.

11. The composition of claim 1, in which the phenol oxidizing enzyme system is a laccase or a laccase related enzyme together with oxygen.

12. The composition of claim 11, in which the laccase is a microbial laccase.

13. The composition of claim 12, wherein the laccase is derived from Coprinus, Myceliophthora, Polyporus, Pycnoporus, Scytalidium or Rhizoctonia.

14. The composition of claim 13, wherein the laccase is derived from Coprinus cinereus, Myceliophthora thermophila, Polyporus pinsitus, Pycnoporus cinnabarinus, Scytalidium thermophilum or Rhizoctonia solani.

15. The composition of claim 1, wherein said composition is an aqueous composition.

16. The composition of claim 15, wherein the concentration of the phenol oxidizing enzyme is in the range of from 0.001-10 mg enzyme protein per liter.

17. The composition of claim 15, wherein the concentration of the enhancing agent corresponds to 1-1000 μM.

18. The composition of claim 1, wherein said composition is a granulate.

19. A method for killing or inhibiting microbial cells comprising treating said microbial cells with the composition of claim 1.

20. A detergent composition comprising a surfactant and the composition of claim 1.

21. A method of inhibiting microorganisms present in laundry, comprising treating the laundry with a soaking, washing or rinsing liquor comprising the composition of claim 1.

22. A method of preserving a cosmetic product, comprising adding an effective amount of the composition of claim 1 to the cosmetic product.

23. The method of claim 22, wherein the cosmetic product is a mouth wash composition, a cosmetic liquid or gel or paste, an eye lotion, an antiperspirant, a deodorant, a nasal spray, an eye ointment, an ointment or cream, a foot bath salt.

24. Use of the composition of claim 1 for cleaning or disinfection of contact lenses.

25. A method of cleaning, disinfecting or inhibiting microbial growth on a hard surface, wherein the surface is contacted with the composition of claim 1.

26. The method of claim 25, wherein the hard surface is a process equipment such as a member of a cooling tower, a water treatment plant, a dairy, a food processing plant, a chemical or pharmaceutical process plant.

27. The method of claim 25, wherein the hard surface is a surface of water sanitation equipment.

28. The method of claim 25, wherein the hard surface is a surface of equipment for paper pulp processing.

29. Use of the composition of claim 1 in a cleaning-in-place system.

30. Use of the composition of claim 1 for disinfection of water systems.

31. The use of claim 30 for disinfection of water systems in paper pulp processing.

32. Use of the composition of any of claims 1-6 for preserving paint.
